(19) United States
(12) Patent Application Publication
(10) Pub. No.: US 2013/0039868 A1
(43) Pub. Date: Feb. 14, 2013

(54) Dentifrice Composition

(71) Applicant: Tracy Kean, Kelseyville, CA (US)

(72) Inventor: Tracy Kean, Kelseyville, CA (US)

(21) Appl. No.: 13/571,333

(22) Filed: Aug. 9, 2012

Related U.S. Application Data
(60) Provisional application No. 61/522,105, filed on Aug. 10, 2011.

(51) Int. Cl. A61K 8/97; A61K 8/19; A61Q 11/00; A61K 8/92

(52) U.S. Cl. 424/53; 424/49; 424/58

(57) ABSTRACT

A teeth whitening dentifrice that enhances the shades of teeth with only a short application on the teeth. The dentifrice disrupts the surface tension between the teeth and the stains to provide with the aid of commonly known mechanical brushing and rising methods. 1 to 3 shades of whiter teeth. The toothpaste combines a non-polar solvent, a polar solvent, and a surfactant. An emulsion creates electrostatic interactions with the polar solvent and a reverse micelle to create a hydrophobic compound, which combines with the non-polar solvent to create a non-polar environment on the teeth. The polar solvent's strong electrostatic interactions in reverse micelles accelerate the removal of soluble material. The non-polar environment bonds with insoluble materials, such as extrinsic stains, to disrupt the surface tension between the teeth and the stain. Bleaching agents, thickeners, polishing agents, and other enhancers are mixed to the toothpaste to provide additional functions and characteristics.

### CROSS- REFERENCE TO RELATED APPLICATIONS

The present Utility patent application claims priority benefit of the U.S. provisional application for patent Ser. No. 61522105 entitled “COSMETIC TOOTHPASTE/GEL”, filed on 10-Aug.-2011, under 35 U.S.C. 119(e). The contents of this related provisional application are incorporated herein by reference for all purposes to the extent that such subject matter is not inconsistent herewith or limiting hereof.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to toothpaste. More particularly, one or more embodiments of the invention relate to whitening toothpaste.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that teeth are small, calcified, whitish structures found in the mouth that are used to break down food. Teeth are made of multiple tissues of varying density and hardness.

Typically, oral hygiene is the practice of keeping the teeth clean and is a means of preventing dental caries, gingivitis, periodontal disease, bad breath, and other dental disorders. Regular cleanings, usually done by dentists and dental hygienists, remove tartar that may develop even with careful brushing and flossing. A toothbrush is able to remove most plaque, except in areas between teeth. As a result, flossing is also considered a necessity to maintain oral hygiene.

Typically, tooth whitening, is a common procedure in general dentistry. One can expect that as a person ages the adult teeth often become darker due to changes in the mineral structure of the teeth, as the enamel becomes less porous. Teeth can also become stained by bacterial pigments, food-goods, and tobacco.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary first dentifrice composition, in accordance with an embodiment of the present invention;

FIG. 2 illustrates an exemplary second dentifrice composition, in accordance with an embodiment of the present invention; and

FIG. 3 illustrates an exemplary scale containing a row of teeth progressing from an initial shade to an enhanced shade, in accordance with an embodiment of the present invention.

Unless otherwise indicated illustrations in the figures are not necessarily drawn to scale.

### DETAILED DESCRIPTION OF SOME EMBODIMENTS

Embodiments of the present invention are best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

The present invention will now be described in detail with reference to embodiments thereof as illustrated in the accompanying drawings.

In one embodiment of the present invention, a dentifrice composition 100 may provide a teeth whitening dentifrice that enhances the shades of teeth 302 with only a short application on the teeth. The dentifrice may disrupt the surface tension between the teeth and insoluble materials 302 to provide 1 to 3 shades of whiter teeth. In some embodiments, the dentifrice composition may combine a non-polar solvent, a polar solvent, and a surfactant. An emulsion creates electrostatic interactions with the polar solvent and a reverse micelle to create a hydrophobic compound, which combines with the non-polar solvent to create a non-polar environment on the teeth. The polar solvent's strong electrostatic interactions in reverse micelles may accelerate the removal of soluble material. The non-polar environment bonds with insoluble materials, such as extrinsic stains, to disrupt a surface tension between the teeth and the stain. Bleaching agents, thickeners, polishing agents, flavoring agents, tetrasodium pyrophosphate salt, titanium dioxide, and other enhancers may be mixed to the toothpaste to provide additional functions and characteristics. In this manner the dentifrice composition may be helpful for removing the insoluble material from the teeth. The insoluble material may include, without limitation, extrinsic stains, intrinsic stains, plaque, calculus, polar ionicly bonded material, and non-polar ionicly bonded material. In some embodiments, the extrinsic stains may contribute to off colored teeth. The extrinsic stains may include, without limitation, tobacco, coffee, red wine and other product staining material.

Those skilled in the art, in light of the present teachings, will recognize that the combined effects of a non-polar solvent and a polar solvent mixed with a dual surfactant, may create a soluble composition that instanly effects the surface tension between the pellicle film and insoluble materials. In this manner, the nonpolar environment created by the dentifrice composition may more effectively bond with insoluble materials on the teeth, thereby disrupting the surface tension between the pellicle film and the insoluble materials.

In one embodiment of the present invention, the non-polar solvent may include oil. The oil may include, without limitation, Canola oil, vegetable oil, and wax. In some embodiments, the oil may include a range of about 0.1% to 99% at concentration of about 30% by weight of the dentifrice composition. Those skilled in the art, in light of the present teachings, will recognize that non-polar solvents dissolve non-polar compounds best, since like dissolves like. Further, strongly non-polar compounds, including, without limitation oils or waxes, dissolve only in non-polar organic solvents, including, without limtiation, the insoluble materials.

In one embodiment of the present invention, the polar solvent may include water and/or water based compounds, including, without limitation, glycerin. In one embodiment, the polar solvent may include, without limitation an Aqua phase. The Aqua phase may include a concentration of about 0.1% to 99% by weight of the dentifrice composition. However, in one embodiment, the water may include a concentration of about 54.5% by weight of the dentifrice composition. Those skilled in the art, in light of the present teachings, will recognize that the oxygen atom in a water molecule has a greater affinity for proximate electrons than the hydrogen atom. This allows the water molecule to create an asymmetrical distribution of charge. This polar property allows the water to separate like, polar solute molecules, thereby completing the reverse soluble emulsion. The water's strong electrostatic interactions in reverse micelles accelerate the removal of soluble material. Other material that may be used as polar solvents may include, without limtiation, tap water, spring water, distilled water, and flavored water.

In one embodiment, the water may combine with a humectant to form the Aqua phase. Those skilled in the art, in light of the present teachings will recognize that the humectant may help blend the dentifrice composition, and also help to adhere the dentifrice composition to the teeth. In some embodiments, the humectant may include, without limtiation, glycerin. Glycerin may include a simple polyol compound (C3H803). The glycerine may be included in the dentifrice composition at a concentration range from about 0.1 to 99% by weight. However, in other embodiments, the glycerin may include about 32.5% by weight of the dentifrice composition. Those skilled in the art, in light of the present teachings will recognize that glycerin may be a odorless, neutral, sweet-tasting, colorless, thick liquid which freezes to a gummy paste and which has a high boiling point. Glycerin may be dissolved into water or alcohol, but not in non-polar solvents like oils. However, in some embodiments, many things may dissolve into glycerin easier than into water or alcohol, thereby providing an effective solvent. Those skilled in the art, in light of the present teachings, will recognize that glycerol may reduce the coefficient of friction of polymer coated surfaces by several orders of magnitude. This effect may be attributed to the enhanced viscosity of glycerol-water solutions as compared to pure water. Other agents may also be employed for the dentifrice composition including, without limtiation, glycerin, sorbitol, polyethylene glycol, and any other suitable polar solvent.

In one embodiment of the present invention, the duel surfactant may include sodium lauryl sulfate. The sodium lauryl sulfate may include a concentration of about 0.1% to about 2% by weight of the dentifrice composition. However, in one embodiment, the sodium lauryl sulfate may include a concentration of about 0.5% by weight of the dentifrice composition. Those skilled in the art, in light of the present teachings, will recognize that sodium lauryl sulfate is an organic compound. The salt is of an organosulfate consisting of a 12-carbon tail attached to a sulfate group, giving the material the amphiphilic properties required of a detergent derived from oils, including, without limtiation, coconut oil and palm oil. Surfactant molecule may contains either a water insoluble or oil soluble component, and a water soluble component. Surfactants may be organic compounds that are amphiphilic, including, without limtiation, both hydrophobic groups (tails), and hydrophilic groups (heads).

The synergy created between the combination of non-polar solvent, polar solvent, and duel surfactant may enhance the cleaning and whitening effect of the dentifrice composition, and instantly disrupt the surface tension between the teeth and the insoluble material. In some embodiments, the disruption in the surface tension may result in an instant visible color change of 1 to 3 shades in about two minutes. The color change may result in the revelation of the natural/original color of the teeth and both natural and restorative material.

In one embodiment of the present invention, the dentifrice composition may also minimize the required exposure time between the dentifrice composition and the teeth for producing results. Those skilled in the art, in light of the present teachings, will recognize that irritation to the teeth may be reduced because the the teeth whiten from an initial shade to an enhanced shade in an expedited timeframe. In a further embodiment, the irritation in the teeth may be reduced because of the lack of bleaching agents and harsh abrasives in the dentifrice composition.

In one embodiment of the present invention, the dentifrice composition may reduce the amount of time that the teeth are in contact with the cleaning agents, thereby minimizing sensitivity in the teeth. In one embodiment, the present invention may not include bleachings agents. The lack of bleaching agents may further reduce sensitivity in the teeth from using the dentifrice composition. The amount of time that the dentifrice composition is in contact with the teeth, and the allowable range of each compound in the dentifrice composition may provide enhanced teeth whitening effects in relation to a commercial dentifrice. The following table illustrates the difference allowable ranges between the present invention and exemplary commercial dentifrice:

| | Percentage | | | |
|---|---|---|---|---|
| | Allowable Range “emulsion” | | | Allowable Range |
| | #1 | #2 | #3 | #4 |
| Humectants | 42.5% | 12% | 4.7% | (0.1% to 99%) |
| Non Polar solvent | 45% | 45% | 76% | (0.1% to 99%) |
| Water | 12% | 42.5% | 15% | (0.1% to 99%) |
| Emulisfier | 0.5% | 0.5% | 4.30% | (.0001 to 10%) |
| Total Base | 100% | 100% | 100% | |

| | Percentage | | | |
|---|---|---|---|---|
| | Allowable Range emulsion | | | |
| | in a commercial dentifrice | | | |
| | #1 | #2 | #3 | |
| Humectants | 31.6% | 30% | 47% | (0.1% to 99%) |
| Water | 7% | 2.1% | 2% | (0.1% to 99%) |
| Canolia oil | 30% | 25% | 40% | (0.1% to 99%) |
| Abrasive Hydrated | 24.5% | 39.4% | 5% | (.0001 to 75%) |
| silica | | | | |
| Sodium Bicarbonate | 3.5% | 0.0 | 0.0% | (0 to 75%) |
| Sodium lauryl Sulfate | 0.50% | 0.50% | 0.40% | (.0001 to 10%) |
| | | | | 5.9% |
| Sodium hydroxide | 0.60% | 0.60% | 0.60% | (0 to 2%) |
| Sodium Sacciran | 0.40% | 0.40% | 0.40% | (0 to 2%) |
| Tetrasodium | 0.50% | 0.50% | 0.50% | (0 to 2%) |
| Pyrohospate | | | | |
| Flavor (mint) | 0.50% | 0.50% | 0.50% | (0 to 5%) |
| Xanthan Gum | 0.50% | 0.50% | 1.60% | (0 to 5%) |
| Cellulose Gum | 0.0% | 0.50% | 1.70% | (0 to 10%) |
| Calcium peroxide | 0.0% | 0.0% | 0.0% | (0 to .4999%) |
| Preservative | 0.40% | 0.40% | .4% | (0 to 1%) |
| (s. quantity) | | | | |
| Titanium dioxide | 0.6% | 0.6% | 0.0% | (0 to 5%) |
| Total Base | 100% | 100% | 100% | |

In one embodiment of the present invention, the dentifrice composition may include various forms for dispersion and application on the teeth, including, without limitation, emulsions, creams, pastes, gels, rinses, aerosol spray, and other suitable delivery system. In some embodiments, the dentifrice composition may be utilized on natural teeth, false teeth, crowns, bridges, and polymer filling of teeth. In one embodiment, the dentifrice composition may include a pH of about 8.

In some embodiments, the dentifrice composition may utilize the aid of mechanical and rinsing methods to disrupted and dislodged material in the teeth, including, without limitation, a toothbrush, dental floss, a dental pick, a water pick, a heating device and dentistry tools. In this manner, the dentifrice composition may include an enhanced ability to improve the overall look and feel of the surface of the teeth, and reveal the natural color of the teeth.

In one embodiment of the present invention, the dentifrice composition may include an emulsion that is efficacious for whitening, cleaning, and polishing teeth. The emulsion may help to remove water insoluble material, and water soluble material in proximity to a gum line, including, without limitation a supragingival. In some embodiments, the dentifrice composition may form an inverse emulsion. The inverse emulsion may include a strong electrostatic interaction with polar particles. The inverse emulsion may further include strong electrostatic interactions in reverse micelles that enhance engagement with the pellicle film and accelerate the removal of the insoluble material.

In some embodiments, the emulsion may include properties demonstrating a strong electronic interaction, of a reverse micelle (hydrophobic head) at the core. The emulsion may interact in a fairly balanced but dominant aqueous phase of about 40% by weight (hydrophonic head), a hydrophonic phase 31.5% by weight, and an abrasive material of about 28% by weight, with an emulsifier for providing stability at about 0.5% by weight. The emulsion may form a first electrostatic interaction with the polar solvent, and a a second electrostatic interaction with a reverse micelle. The interactions are efficacious for disrupting the surface tension between the insoluble material and a soluble material from the pellicle film of the teeth.

In one embodiment of the present invention, a surfactant 104 may be included in the dentifrice composition for providing thorough dispersal of the dentifrice composition throughout the teeth when applied thereto, as well as to improve the cosmetic acceptability and detersive and foaming properties of the dentifrice composition. In some embodiments, the dentifrice composition may include a duel surfactant at concentration range of about 0.1% to 2% by weight, a non-polar solvent at a concentration range of about 0.1% to 99% by weight, and a water based compound at water ratio of 0.1% to 99% by weight. The surfactant may include a concentration of about 0.001 to about 2.0% by weight. However, in other embodiments, the surfactant may be about 0.5 to about 1.0% by weight.

In some embodiments, the organic surfactants efficacious in the practice of the present invention may include salts of the higher alkyl sulfates, including, without limitation, sodium lauryl sulfate (SLS) or other suitable alkyl sulfate having 8 to 18 carbon atoms in the alkyl group; sodium lauryl sulfoacetate, salts of sulfonated monoglycerides of higher fatty acids, such as sodium coconut monoglyceride sulfonate or other suitable sulfonated monoglycerides of a fatty acids of 10 to 18 carbon atoms; salts of amides of higher fatty acid, e.g., 12 to 16 carbon atom acids, with lower aliphatic amino acids, such as sodium-N-methyl-N-palmitoyl tauride, sodium N-lauroyl-, N-myristoyl- and N-palmitoyl sarcosinates; salts of the esters of such fatty acids with isothionic acid or with glycerol monosulfate, such as the sodium salt of monosulfated monoglyceride of hydrogenated coconut oil fatty acids; salts of olefin sulfonates, e.g. alkenes sulfonates or hydroxalkene sulfonates or mixtures thereof having 12 to 16 carbon atoms in the carbon chain of the molecule; and soaps of higher fatty acids, such as those of 12 to 18 carbon atoms, e.g., coconut fatty acids. In one embodiment, the cation of the salt may be sodium potassium or mono-, di or triethanol amine.

Those skilled in the art, in light of the present teachings, will recognize that when the surfactants agglomerate with oil, the resultant aggregate may include a reverse micelle. In a reverse micelle, the heads are in the core and the tails maintain favorable contact with the oil, exposing the hydrophobic cleaning agents for better contact with the teeth. This results in disruption between the surface tension of the insoluble material and the teeth. In yet another embodiment, cleaning agents in the hydrophobic groups and the non-polar solvent (canola oil) may be combined to create a synergy that enhances the cleaning effect. A calcium phosphate salt may dissolve on contact with the teeth, breaking the covalent VB bond, thereby releasing the insoluble material from the teeth.

Those skilled in the art, in light of the present teachings, will recognize that the oil to water ratio may be inherent to removing the insoluble material from the teeth. The higher the concentration of non-polar solvent (canola oil), the more effective the surfactant is in breaking up the surface tension between the insoluble material and the teeth.

In one embodiment of the present invention, a polishing agent 106 may be incorporated in the dentifrice composition for polishing, and helping to whiten the teeth. Those skilled in the art, in light of the present teachings, will recognize that the polishing agent may include, without limitation, a mild abrasive. The abrasive may include micro granules sized and dimensioned to engage crevices and recesses in the teeth. Suitable polishing agents may include, without limitation, siliceous materials, such as silica, and will normally have a mean particle size up to about 10 microns and a very high surface area, e.g. in the range of 150-750 square meters/gram. A preferred silica is a precipitated amorphous hydrated silica, such as silicas (CST), or Sorbosil AC-35 marketed by Crosfield Chemicals, sodium bicarbonate, but other polishing agents may also be employed, including, Zeodent 115 from J. M. Huber Company, calcium carbonate, sodium metaphosphate, potassium metaphosphate, tricalcium phosphate, calcium pyrophosphat calcium phosphate dihydrate, anhydrous dicalcium phosphate, magnesium orthophosphate, trimagnesium phosphate, alumina trihydrate, aluminum silicate, zirconium silicate, calcined alumina and bentonite.

The polishing agent may be present in the dentifrice composition at a concentration of about 0% to about 60% by weight. However, in other embodiments, polishing agent may be present in the dentifrice composition at a concentration of about 25% to about 50% by weight in paste form and 0 to 10% by weight in gel form.

Those skilled in the art, in light of the present teachings, will recognize that hydrated silica, when included in the dentifrice composition may be included at a concentration of about 0% to about 60% by weight, or in other embodiments, at a range of about 20 to 45% by weight in paste form. In yet another embodiment, bicarbonate compounds, when included in the dentifrice composition may be included at a concentration of about 0% to about 60% by weight depending on the delivery system used, and in paste form at a range of about 10% to 40% by weight.

In one embodiment of the present invention, a thickener 108 may be included in the dentifrice composition for providing texture to the dentifrice composition. The thickener may include an organic thickener and/or an inorganic thickener. The inorganic thickener may include fumed silicas such as Cab-o-sil available from Cabot

Corporation, and thickening silicas including those available from W. R. Grace designated Sylox 15. In yet another embodiment of the present invention, the organic thickener may be incorporated with the dentifrice composition. Those skilled in the art, in light of the present teachings, will recognize that organic thickeners such as natural and synthetic gums and colloids may be incorporated in the dentifrice composition of the present invention, particularly when water in amounts up to about 9% by weight are present in the dentifrice composition. Examples of organic thickeners may include, without limitation, carrageenan (Irish moss), xanthan gum and sodium carboxymethyl cellulose, starch, polyvinylpyrrolidone, hydroxyethylpropylcellulose, hydroxybutyl methyl cellulose, hydroxypropyl methyl cellulose, and hydroxyethyl cellulose. The inorganic or organic thickener may be incorporated in the dentifrice composition at a concentration of about 0.05 to about 2% by weight. In yet another embodiment, the inorganic or organic thickener may be incorporated in the dentifrice composition at a concentration of about 0.1 to about 1.5% by weight.

In one embodiment of the present invention, a peroxide compound 110 may be used as an ingredient in the dentifrice composition as a bleaching agent. The peroxide compound may be present in the dentifrice at a concentration of about 0 to about.499% by weight. Suitable peroxide compounds may include metal peroxides, including, without limitation, carbamide peroxide, calcium peroxide, magnesium peroxide and zinc peroxide. In some embodiments, a calcium peroxide compound may be used as an ingredient in the dentifrice composition. The calcium peroxide may be present in the dentifrice at a concentration of about 0 to about 0.499% by weight, or about 0.25 to about 0.499% by weight. In some embodiments, the calcium peroxide may be used as a whitening agent, and may be incorporated in the dentifrice composition at concentration range at of about 0 to 0.499% by weight at a range of about 0.25% to 0.499% by weight. In one alternative embodiment, a bleaching agent may be employed. The bleaching agent may be present in the dentifrice composition at a concentration of about 0.3% to 0.499% by weight.

In one embodiment of the present invention, tetrasodium pyrophosphate salts may be incorporated in the dentifrice composition at the concentration range of about 0.5% to 2% by weight. In some embodiments, the tetrasodium pyrophosphate salts may be utilized as a retardant for preventing insoluble material that has disengaged from the pellicle film of the teeth from resettling on the teeth.

In one embodiment of the present invention, the dentifrice composition may include titanium dioxide to provide color to the dentifrice composition. The titanium dioxide may be used to maintain oil to base stability. In the present invention titanium dioxide may be used to provide whitening properties. The titanium dioxide may be incorporated in the dentifrice composition at a concentration range of about 0.3 to 2%. However, in one embodiment, the dentifrice composition at a concentration includes about 0.7% by weight.

Those skilled in the art, in light of the present teachings, will recognize that preservatives may be added to the dentifrice composition since the moisturizer and certain mucilages favor the development of microbial flora. The preservatives that may be utilized may include, without limitation, benzoic acid, formol, and phenol.

In one alternative embodiment, a flavoring composition may also be incorporated in the dentifrice composition. Suitable flavoring compositions may include, without limitation, flavoring oils, e.g., oils of spearmint, peppermint, wintergreen, sassafras, clove, sage, eucalyptus, marjoram, cinnamon, lemon, and orange, and methyl salicylate. Suitable sweetening agents include sucrose, lactose, maltose, sorbitol, xylitol, sodium cyclamate, perillartine, and sodium saccharin. In one embodiment, the flavoring compositions may together comprise from 0.01% to 5% or more of the dentifrice composition.

In one embodiment of the present invention, the dentrifrice composition may be prepared, without limtiation, in the following manner:
1. Dissolve sodium bicarbonate in 100% of water.
2. Separately, mix the humectants (e.g. glycerin and glycol humectants) in a conventional mixer, until they are dispersed and until the mixture becomes a homogeneous gel phase.
3. Into the gel phase add the anticalculus agents. Mix these ingredients until a homogeneous phase is obtained.
4. Mix the water/sodium bicarbonate with the thickeners, polishing agent, flavoring composition and surfactant ingredients. Mixed at high speed under vacuum of about 20-100 mm Hg.
5. At this time the non-polar solvent, including, without limitation, canola oil is added mixed at high speed under vacuum of about 20-100 mm HG. The resultant product is a homogeneous, semi-solid, extrudable paste product.

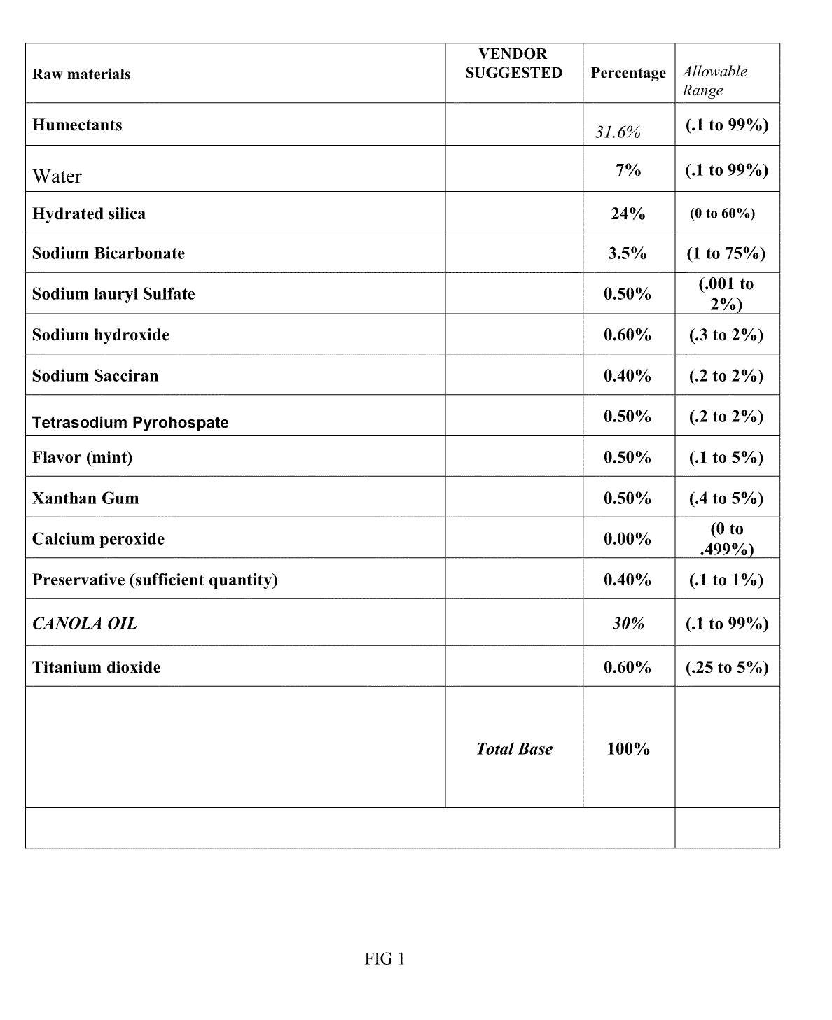

FIG. 1 illustrates an exemplary first dentifrice composition 100, in accordance with an embodiment of the present invention. In the present embodiment, the first dentifrice composition may be utilized to enhance the color of teeth from the initial shade to the enhanced shade. The dentifrice composition of the present embodiment may be utilized for home use because of the enhanced taste and texture. However, either the first dentifrice composition or the second dentifrice composition may be utilized for professional or home use.

The first dentifrice composition may be prepared in the following manner:
1. Dissolve sodium bicarbonate in 100% of water.
2. Separately, mix the humectants e.g. glycerin and glycol humectants and sorbitol, are dispersed in a conventional mixer until the mixture becomes a homogeneous gel phase.
3. Mix the ingredients until a homogeneous phase is obtained.
4. Mix the water/sodium Bicarbonate with the thickener, polishing agent, reactive ingredient such as peroxide, flavor and surfactant ingredients are added and the ingredients mixed at high speed under vacuum of about 20-100 mm Hg.
5. At this time the non-polar solvent (canola oil) is added and mixed at high speed under vacuum of about 20-100 mm HG.

In one embodiment of the present invention, the method for applying the dentifrice composition to the teeth may include, without limtiation:
1. Applying about 1 milliliter of the dentifrice composition to any form of toothbrush and/or dental surface cleaning devise.
2. Using with normal action covering all exposed surfaces of the teeth for 2 minutes.

Those skilled in the art, in light of the present teachings will recognize that the recommended use is once a day for up to 14 days then a maintance program of 2 to 4 times a month. This may be followed by continued brushing with regular fluoride toothpaste twice a day and visits to a dentist twice a year.

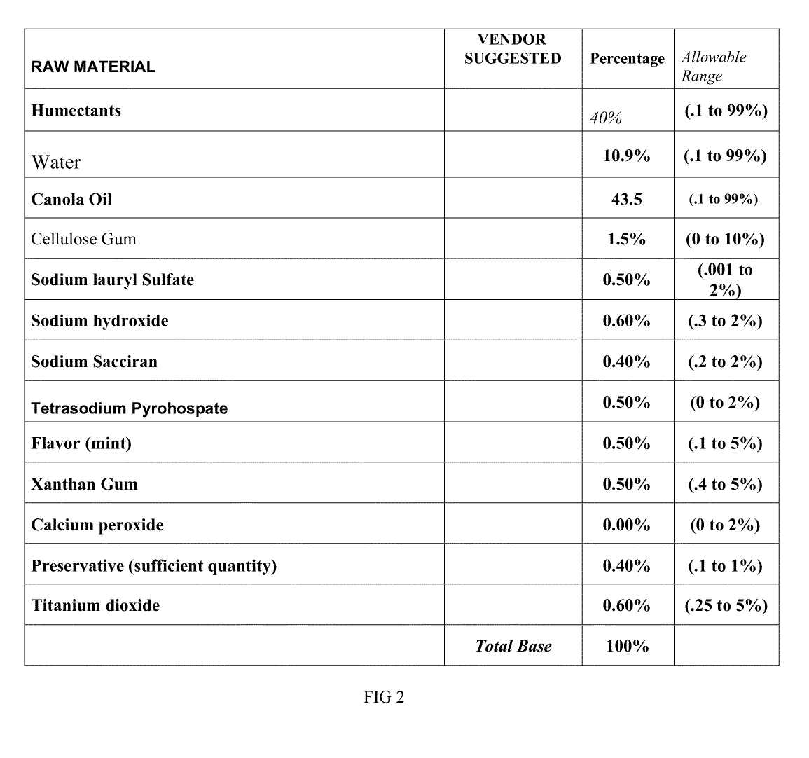

FIG. 2 illustrates an exemplary second dentifrice composition 200, in accordance with an embodiment of the present invention. In the present embodiment, the second dentifrice composition may not include the polishing agent, like in the first dentifrice composition. However, the second dentifrice composition may include a non-polar solvent 202, a surfactant 204, a thickener 208, and a peroxide compound 210, similar to the first dentifrice composition. The second dentifrice composition may also include a greater amount of humectant than the first dentifrice composition for absorbing water. The dentifrice composition of the present embodiment may be utilized in a dental office, for professional products (this should be for FIG. 2)

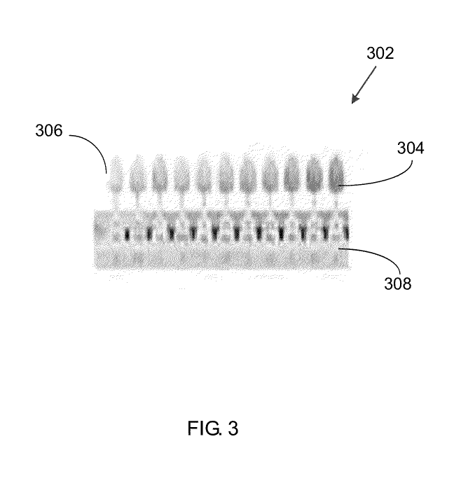

FIG. 3 illustrates an exemplary scale containing a row of teeth progressing from an initial shade to an enhanced shade, in accordance with an embodiment of the present invention. In the present embodiment, the dentifrice composition may whiten the teeth from the initial shade 304 to the enhanced shade 306 in an expedited timeframe. The teeth may appear to lose the insoluble material and change from a darker shade to a lighter shade along the gradient scale 308. In the present embodiment, each simulated tooth is a progressively darker shade on the scale from 0 to 4. The final color of the teeth may include a bright, white color. The scale may be utilized as a visual illustration of the progressive enhancement of the teeth. The scale may range from 0, which represents the most whiteness, to 4, which represents the darkest teeth with the most insoluble materials bonded to the teeth. However, in other embodiments, a scale with greater or lesser levels of shading may be utilized. In one embodiment of the present invention, the enhanced shade may be substantially realized when the dentifrice composition is utilized once a day for up to 14 days, followed by a maintance program of 2 to 4 times a month. This may be followed by continued brushing with regular fluoride toothpaste twice a day and visits to a dentist twice a year.

In one embodiment of the present invention, an experiment may be performed to assess the efficacy of the dentifrice composition among a multiplicity of subjects. The expirement may measure the before and after whiteness of the teeth, whereby the teeth may include less insoluble materils after utilizing the dentifrice composition. In one embodiment, the conclusion of the experiment may conclude that the teeth progress from an initial shade having large quantities of insoluble materials on the teeth, to an enhanced shade having less insoluble materials on the teeth. One embodiment of the experiment may include, without limitation, a photographic comparison showing before and after photographs of the visual color change in the teeth. Each subject may be photographed before use of the dentifrice composition. Then, each subject may be given a toothbrush with 0.1 milliters of the dentifrice composition. Then the teeth may be engaged with a toothbrush for 2 minutes.
SUBJECT: A=Earl
SUBJECT: B=Sara
SUBJECT: C=Bob

Utilizing the scale as a color guide, the shades from the experiment may range from M1 (0) A 3 SHADE CHANGE FROM
SUBJECT: (A) BEFORE: M2 (1) TO AFTER M1 (1)=A 3 SHADE CHANGE
SUBJECT: (B) BEFORE: M1.5 (1) TO AFTER M1 (1)=A 1 SHADE CHANGE.
SUBJECT: (C) BEFORE: M2 (3) TO AFTER M1 (1.5)=A 6 SHADE CHANGE.

Those skilled in the art, in light of the present teachings will recognize that increasing the oil content of the dentifrice composition may enhances the color of the teeth. Increasing the timeframe for utilizing the dentifrice composition may also provide an enhanced shade. Further, combining mechanical cleaning with the dentifrice composition may also enhance the efficacy.

In one alternative embodiment of the present invention, the dentifrice composition may include various concentrations of non-polar solvents for controlling the enhancement of the teeth. In yet another alternative embodiment of the present invention, the dentifrice composition may be utilized to emulsify insoluble materials in industrial machines by increasing the composition of the non-polar compound or oil content.

Some embodiments of the present invention may also provide the capability of a brushing and/or buffing or rinsing action to most, if not all, the exposed teeth surfaces by conventional means such as, but not limited to, a toothbrush (e.g., a bristle attach to a handle) and/or buffer such as, but not limited to, non toxic, rubbers, natural and/or synthetic cloths, any scrubbing tools in any suitable variety of shapes that sufficiently conform to the area being cleaned. I some implementations, application may be done with, or as if, by motions, of either manual and/or motorized operation of brush and/or buffer. Similarly, removal may be done, or as if, by motions of a brush or buffer. In many practically applications, the present brushing application embodiment enables working in the material so that is does not just sit on the tooth, and, likewise, this brushing action assists material removal. Those skilled in the art will readily recognize, in light of the teachings of the present invention, alternative and suitable techniques for applying and/or removing the material depending on the needs of the particular application.

All the features or embodiment components disclosed in this specification, including any accompanying abstract and drawings, unless expressly stated otherwise, may be replaced by alternative features or components serving the same, equivalent or similar purpose as known by those skilled in the art to achieve the same, equivalent, suitable, or similar results by such alternative feature(s) or component(s) providing a similar function by virtue of their having known suitable properties for the intended purpose. Thus, unless expressly stated otherwise, each feature disclosed is one example only of a generic series of equivalent, or suitable, or similar features known or knowable to those skilled in the art without requiring undue experimentation.

Having fully described at least one embodiment of the present invention, other equivalent or alternative methods of implementing a non-polar environments for removing insoluble materials from teeth according to the present invention will be apparent to those skilled in the art. Various aspects of the invention have been described above by way of illustration, and the specific embodiments disclosed are not intended to limit the invention to the particular forms disclosed. The particular implementation of the non-polar environments for removing insoluble materials from teeth may vary depending upon the particular context or application. By way of example, and not limitation, the non-polar environments for removing insoluble materials from teeth described in the foregoing were principally directed to removing extrinsic stains from teeth with a dentifrice composition implementations; however, similar techniques may instead be applied to removing insoluble materials from industrial machines by increasing the percent composition of the non-polar compound, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. A dentifrice composition comprising:
a non-polar solvent, said non-polar solvent comprising about 0.1% to 99% by weight of said dentifrice composition, said non-polar solvent being operable to disrupt a surface tension between an insoluble material and a pellicle film;
a polar solvent, said polar solvent comprising about 0.1% to 99% by weight of said dentifrice composition, said polar solvent being configured to mix with said non-polar solvent, said polar solvent being operable to enhance the disruption of said surface tension between said insoluble material and said pellicle film;
a surfactant, said surfactant comprising about 0.001% to 2% by weight of said dentifrice composition, said surfactant being operable to disperse said dentifrice composition on teeth; and
an emulsion, said emulsion being operable to form a first electrostatic interaction with said polar solvent, said emulsion further being operable to form a second electrostatic interaction with a micelle, said emulsion being operable to disrupt said surface tension between said insoluble material and said pellicle film, said emulsion further being operable to disrupt said surface tension between a soluble material and said pellicle film.

2. The dentifrice composition of claim 1, in which said non-polar solvent comprises about 30% by weight of said dentifrice composition.

3. The dentifrice composition of claim, 2 in which said non-polar solvent comprises an oil.

4. The dentifrice composition of claim, 3 in which said non-polar solvent comprises Canola oil.

5. The dentifrice composition of claim, 4 in which said polar solvent comprises about 54.5% by weight of said dentifrice composition.

6. The dentifrice composition of claim, 5 in which said polar solvent comprises an Aqua phase, said Aqua phase comprising water and a humectant, said humectant comprising a glycerin.

7. The dentifrice composition of claim 6, wherein said humectant is operable to help mix said dentifrice composition, said humectant being further operable to help adhere said dentifrice composition to said teeth.

8. The dentifrice composition of claim 7, in which said surfactant comprises about 0.5% by weight of said dentifrice composition.

9. The dentifrice composition of claim 8, in which said surfactant comprises a duel surfactant, said duel surfactant comprising sodium lauryl sulfate.

10. The dentifrice composition of claim 9, in which said dentifrice composition comprises a polishing agent, said polishing agent comprising about 10% to 60% by weight of said dentifrice composition, said polishing agent being operable to help whiten said teeth.

11. The dentifrice composition of claim 10, in which said dentifrice composition comprises a thickener, said thickener comprising about 0.05% to 2% by weight of said dentifrice composition, said thickener being operable to provide texture to said dentifrice composition.

12. The dentifrice composition of claim 11, in which said dentifrice composition comprises a peroxide compound, said peroxide compound comprising about 0% to 0.499% by weight of said dentifrice composition, said peroxide compound being operable to help whiten said teeth.

13. The dentifrice composition of claim 12, in which said dentifrice composition comprises a tetrasodium pyrophosphate salt, said tetrasodium pyrophosphate salt comprising about 0.5% to 2% by weight of said dentifrice composition, said tetrasodium pyrophosphate being operable to inhibit said insoluble material from resettling onto said teeth after being removed from said teeth.

14. The dentifrice composition of claim 13, in which said dentifrice composition comprises a titanium dioxide, said titanium dioxide comprising about 0.3% to 2% by weight of said dentifrice composition, said titanium dioxide being operable to provide color to said dentifrice composition.

15. The dentifrice composition of claim 14, in which said dentifrice composition comprises a flavoring composition, said flavoring composition comprising about 0.1% to 5% by weight of said dentifrice composition, said flavoring composition being operable to provide flavor to said dentifrice composition.

16. The dentifrice composition of claim 15, wherein said dentifrice composition is operable to expedite the whitening of said teeth.

17. The dentifrice composition of claim 16, wherein said dentifrice composition is operable to whiten teeth from an initial shade to an enhanced shade.

18. The dentifrice composition of claim 17, wherein said dentifrice composition is operable to help disrupt said insoluble material for providing about 1 to 3 levels of shade enhancements.

19. A method of preparing a dentifrice composition comprising:
Steps for dissolving a sodium bicarbonate in water;
Steps for adding a humectant;
Steps for adding an anticalculas agent;
Steps for mixing until a paste appears;
Steps for adding a surfactant, a thickener, and a polishing agent;
Steps for mixing in a vacuum; and
Steps for adding a non-polar solvent

20. The method of claim 19, further comprising Steps for applying and/or removing said composition.

21. A dentifrice composition consisting of:
a non-polar solvent, said non-polar solvent comprising about 30% by weight of said dentifrice composition, said non-polar solvent further comprising an oil, said oil comprising Canola oil, said non-polar solvent being operable to disrupt a surface tension between an insoluble material and a pellicle film;
a polar solvent, said polar solvent comprising about 54.5% by weight of said dentifrice composition, said polar solvent comprising an Aqua phase, said Aqua phase comprising water, said Aqua phase further comprising a humectant, said humectant comprising glycerin, said polar solvent being configured to mix with said non-polar solvent, said polar solvent being operable to enhance the disruption of the surface tension between said insoluble material and said pellicle film; and
a surfactant, said surfactant comprising about 0.5% by weight of said dentifrice composition, said surfactant further comprising sodium lauryl sulfate, said surfactant being operable to disperse said dentifrice composition throughout teeth;
an emulsion, said emulsion being operable to form a first electrostatic interaction with said polar solvent, said emulsion further being operable to form a second electrostatic interaction with a micelle, said micelle comprising a reverse micelle, said emulsion being operable to disrupt said surface tension between said insoluble material and said pellicle film, said emulsion further being operable to disrupt said surface tension between a soluble material and said pellicle film;
a polishing agent, said polishing agent comprising about 10% to 60% by weight of said dentifrice composition, said polishing agent comprising a hydrated silica, said polishing agent being operable to help whiten said teeth;
a thickener, said thickener comprising about 0.1% to 1.5% by weight of said dentifrice composition, said thickener being operable to provide texture to said dentifrice composition;
a peroxide compound, said peroxide compound comprising about 0.25% to 0.499% by weight of said dentifrice composition, said peroxide compound being operable to help whiten said teeth;
a tetrasodium pyrophosphate salt, said tetrasodium pyrophosphate salt comprising about 0.5% to 2% by weight of said dentifrice composition, said tetrasodium pyrophosphate being operable to inhibit said insoluble material from resettling onto said teeth after being removed from said teeth;
a titanium dioxide, said titanium dioxide comprising about 0.7% by weight of said dentifrice composition, said titanium dioxide being operable to provide color to said dentifrice composition; and
a flavoring composition, said flavoring composition comprising about 0.1% to 5% by weight of said dentifrice composition, said flavoring composition being operable to provide flavor to said dentifrice composition.
